(19) United States
(12) Patent Application Publication
(10) Pub. No.: US 2007/0255182 A1
(43) Pub. Date: Nov. 1, 2007

(54) VESSEL FILL LEVEL INDICATING DEVICE

(76) Inventor: Alton WERNER, Granite Bay, CA (US)

Correspondence Address: STANDLEY LAW GROUP LLP, 495 METRO PLACE SOUTH, SUITE 210, DUBLIN, OH, 43017, US

(21) Appl. No.: 11/695,647

(22) Filed: Apr. 3, 2007

Related U.S. Application Data
(60) Provisional application No. 60/744,166, filed on Apr. 3, 2006.

(51) Int. Cl. A61B 5/00

(52) U.S. Cl. 600/584; 600/576

(57) ABSTRACT

A vessel fill level is qualitatively determined and indicated by a device having a base and a vessel-supporting member that is resiliently suspended inwardly from and above the base. The base has an annular body with at least three stanchions, symmetrically spaced around the annular body, as well as a plurality of radially-inwardly-extending arms. Each stanchion has a vertical slot and each of the arms meet at an axial base member. The vessel support has a plurality of radially-outwardly extending arms and is adapted to support the vessel. A distal end of each of the arms is received and retained in one of the vertical slots, where a spring connects the arm to the base. Means for distinguishing at least two levels of vertical separation between the base and the vessel-supporting member provides a qualitative signal to a display that may be on the base or remote to the device.

### CROSS REFERENCES TO RELATED APPLICATIONS

This application is a non-provisional of U.S. provisional application 60/744,166, filed 3 Apr. 2007, which is incorporated by reference as if fully recited herein.

### TECHNICAL FIELD

The invention, at least with regard to the exemplary embodiments, relates to a device used in association with a product-containing vessel, especially a product contained under pressure. The device operatively engages the vessel to qualitatively determine the amount of product contained therein and to provide a visual indication of the qualitative determination. The invention has particular application with respect to propane tanks, such as those used in association with grills and the like, industrial gas cylinders and beer kegs.

### BACKGROUND OF THE ART

In many situations involving product-containing vessels, particularly cylinders or tanks containing liquefied gases under pressure, it is not especially critical to be able to readily ascertain the exact amount of the product remaining in the vessel. Qualitatively knowing the fill level of the vessel is sufficient to indicate when the vessel is approaching a point at which it will need to be replaced. This allows appropriate warning to the user to obtain a replacement vessel of the product.

An exemplary situation involves a propane tank commonly used with a barbecue grill or similar cooking or heating device, such as a smoker or a turkey fryer. An empty tank weighs about 8.2 kg and, when filled with propane, the tank weighs about 17.8 kg. While a variety of weight- or pressure-sensing means are readily available for monitoring the amount of gas in the vessel, these means have several characteristics that make them unattractive for the required service.

First, many mechanical scales are sensitive to atmospheric conditions, due to the construction required to provide their accuracy, A propane tank should not be stored in a confined space, as any leak of the propane can result in an explosive propane/air mixture. This risk is reduced greatly by allowing at least limited exposure to the elements.

Second, mechanical scales provide far more accuracy than is required. Accuracy comes at a price, and the price usually exceeds the benefit conferred.

Third, the integrity of any piping that communicates a vessel supplying a flammable gas to a combustion system should be maintained as continually as reasonably possible. Any interruptions to the integrity can result in leaks and/or introduction of air into the piping. For this reason, it is not advisable to remove the vessel from the system other than to replace it.

Fourth, pressure sensing means can be used, but these devices generally do not show a noticeable drop in pressure until all or most of the liquid in the vessel has vaporized, meaning that the vessel is virtually empty.

At least two US patents teach devices where a gauge for monitoring the weight of a propane tank is provided. In one of these, a pressure generated on a fluid in a chamber by the weight of the tank is measured and displayed. In the other, the deflection of a spring bearing the weight of the tank is displayed.

Another US patent teaches a strip that is adhesively applied to an external wall of the vessel. Thermochromic means in the strip, especially liquid crystal materials, can sense temperature differences on an exterior surface of the vessel wall, arising due to thermal conductivity differences between the presence of a liquid or a gas on the adjacent interior surface.

For at least these reasons, it is an unmet need of the known prior art to provide an inexpensive qualitative determination of the fill level of a vessel at the point of use and provide a visual indication of the fill level of the vessel upon demand, without requiring that the vessel be disconnected from the piping into which it dispenses its product.

### SUMMARY OF THE INVENTION

This and other advantages of the present invention, at least in relation to the exemplary embodiments, are achieved by a device for measuring and displaying a relative fill level of a vessel. The device has a base and a vessel-supporting member. The base comprises an annular body with at least three stanchions, symmetrically spaced around a circumference of the annular body and a plurality of radially-inwardly-extending arms, each stanchion having a vertical slot and each of the radially inwardly extending arms meeting at an axial base member. The vessel-supporting member has a plurality of radially-outwardly extending arms and a means for supporting a vessel atop the vessel-supporting member, each arm adapted at a distal end thereof to be received and retained in one of the vertical slots. The device also comprises a plurality of means for resiliently supporting the vessel-supporting means radially inwardly from and above the base, the number of resilient support means corresponding to the number of vertical slots. A means for distinguishing at least two levels of vertical separation between the base and the vessel-supporting member is also a part of the device, as is a means for displaying at least two fill levels of the vessel, based upon the vertical separation distinguished.

In some embodiments, the displaying means comprises a switch to selectively make and break an electrical circuit for displaying the fill level measurement.

In some embodiments, each resiliently supporting means comprises a spring, the ends of which connect an interior of one of the stanchions to the distal end of one of the radially-outwardly extending arms.

In many embodiments, one of the stanchions is provided with a surface for at least one of: displaying the fill level of the vessel or connecting a remote fill level display device to the fill level distinguishing means.

In some embodiments, the vessel-supporting means comprises an annular concave surface for receiving a convex surface of the vessel. In other embodiments, the vessel-supporting means comprises a channel for receiving a cylindrical base of the vessel. In some embodiments, both vessel-supporting means are provided.

In many embodiments, each resiliently supporting means comprises a spring, the ends of which connect an interior of one of the stanchions to the distal end of one of the radially-outwardly extending arms.

In some embodiments, the vertical separation distinguishing means comprises a plurality of electrical contacts resiliently positioned at different distances from a contact plate. In some of these embodiments, the displaying means comprises an electrical circuit that is made by each of the each of the electrical contacts and the contact plate through contact, the circuit resulting in illumination of an indicator corresponding to the electrical contact. Preferably, the indicators are light-emitting diodes and the circuit further comprises a touch switch that is normally open and closes to make the circuit only when activated by a user.

### BRIEF DESCRIPTION OF THE DRAWINGS

A better understanding of the exemplary embodiments will be had when reference is made to the appended drawings, wherein identical parts are identified by identical part numbers and wherein:

FIG. 1 is a perspective view of an exemplary embodiment;

FIG. 2 is a top plan view of the FIG. 1 embodiment;

FIG. 3 is a partially cut-away, elevational view of the FIG. 1 embodiment, showing placement of a vessel on the device;

FIG. 4 is a bottom plan view of the FIG. 1 embodiment;

FIG. 5 is an enlarged depiction of the vertical separation distinguishing means; and

FIG. 6 is a schematic depiction of the display circuit used to display the fill level of the vessel.

### DETAILED DESCRIPTION OF AN EXEMPLARY EMBODIMENT

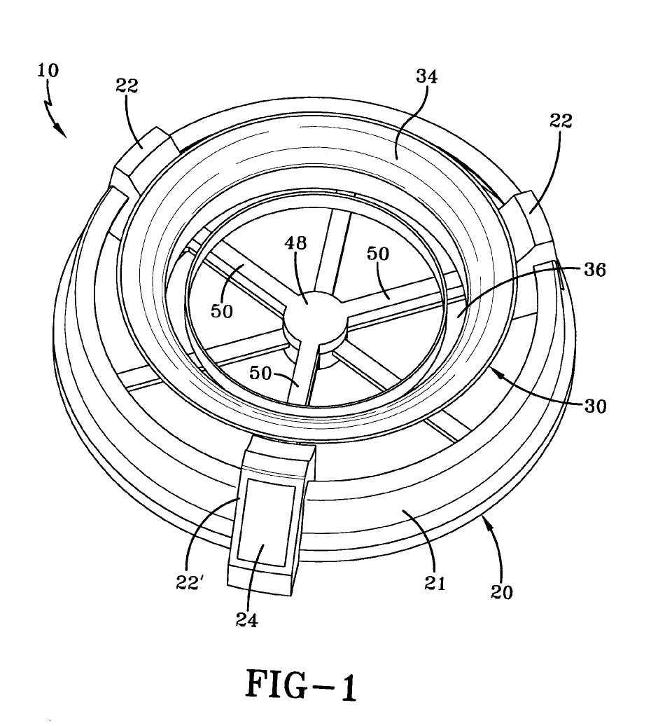

Referring first to FIG. 1, an exemplary embodiment of the device 10 is shown in perspective view, in isolation from a vessel or tank of the type with which it would be used. In the embodiment illustrated, device 10 comprises a base 20 and a vessel-supporting member 30. As will be more readily apparent from a later figure, vessel-supporting member 30 is supported above base 20 at an axial position of the vessel-supporting member and the base. The support is provided such that the weight borne by vessel-supporting member 30 causes it to move vertically relative to the base, particularly at the axial position.

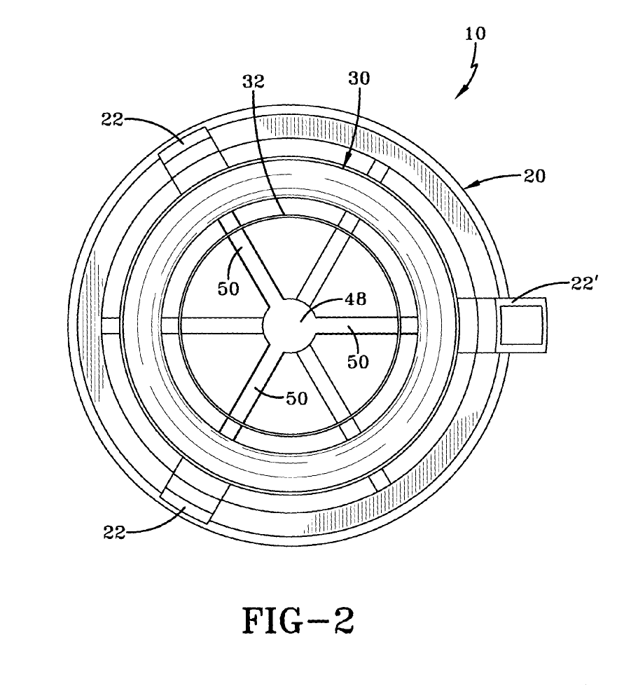

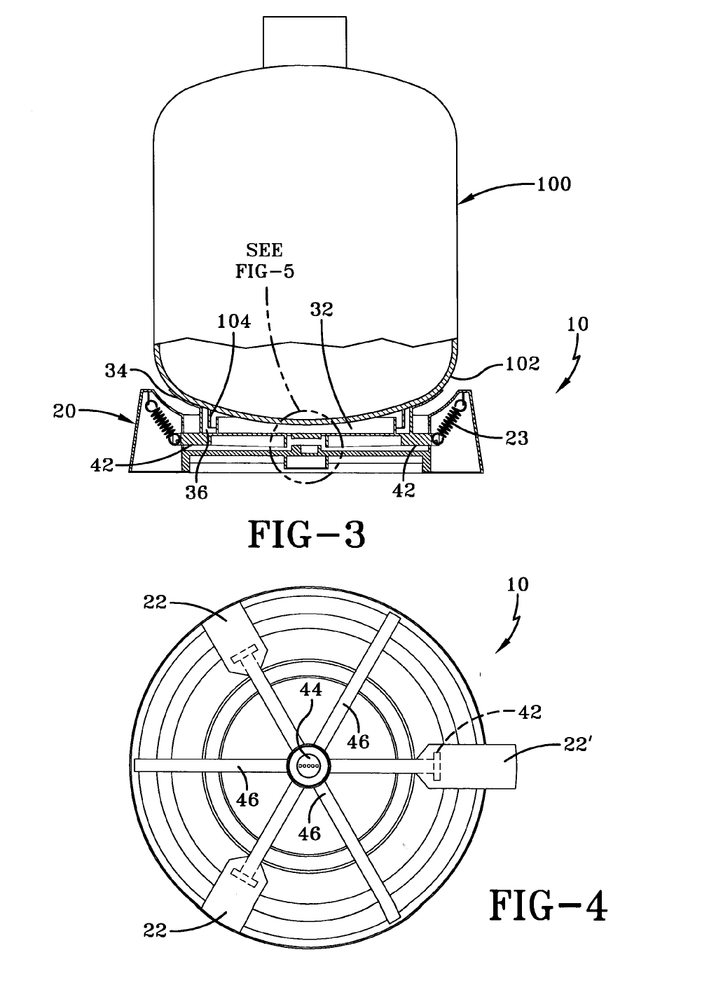

Further features of the device 10 are seen in FIGS. 1-4. Base 20 has an annular body 21. In the exemplary embodiment, annular body 21 has at least three stanchions 22, which are shown as been symmetrically arranged around a circumference of the annular body 21. These stanchions 22, which extend radially inward from base member 20 help to stabilize the position of dish member 30. In the embodiment illustrated, one stanchion 22′ extends radially outwardly in addition to the radially-inward extension. This outward extension provides a surface 24 which may, in some embodiments, visually display a fill level signal generated by the device 10, as explained in more detail below. In other embodiments, surface 24 may be used to provide a point of connection or passage of a signal wire (not shown) to cause a visual display of a fill level signal remote from the base 20. In yet other embodiments, surface 24 not only provides the display, but also provides the connection to a remote display.

Vessel-supporting member 30 also has features apparent in FIGS. 1-4. In the exemplary embodiment shown, member 30 has a generally depressed interior area 32, with a diameter sized to receive a cylindrical bottom portion of the object to be weighed, particularly the bottom of a propane tank 100 as used with a barbecue grill, especially a propane tank with an arcuate bottom surface 102 and a cylindrical base 104. In the illustrated embodiment, two separate means are provided to receive the vessel. The first is an annular concave surface 34 sized and adapted to receive tank bottom surface 102. The second means is an annular channel 36, radially inward from the concave surface 34, the channel 36 being sized and adapted to receive the vessel cylindrical base 104. Interior area 32 is provided with open area to allow free drainage of any moisture, so that the weight sensed by the device 10 is based solely upon the weight from the vessel 100.

Additionally, vessel-supporting member has a plurality of radially-outwardly extending arms 42. One of these arms 42 is provided for each stanchion 22, and the arm is long enough that it can be received into a vertically-extending slot that in provided on a radially-inward face of the stanchion. The arms may be enlarged at their distal ends, to restrain radial movement of the arm within the slot. Further, upward motion of the vessel-supporting member 30 is stopped by impingement of the top of the arm 42 against the top of the slot.

A resilient suspension means, especially a spring 23, and more especially, a coil spring, is used inside each of the generally hollow stanchions 22 to connect the annular body 21 to the arm 42 that extends into the stanchion through the slot. In this manner, the vessel-supporting member 30 is resiliently suspended inwardly from and above the base.

At the axial center of the device 10, each of the base 20 and the vessel-supporting member have a planar portion, the relative vertical separation of these planar portions providing a qualitative indication of the fill level of the vessel placed atop the vessel-supporting member. In the illustrated embodiment, the planar portion on the base 20 is a central base plate 44 that is connected by a plurality of radial arms 46. Typically, these radial arms 46 run from one of the stanchions 22 to the central base plate. Also in the illustrated embodiment, the central planar portion of the vessel-supporting member 20 is a cap 48 that is connected to a plurality of radial arms 50 that run radially inwardly from the annular channel 38. In another embodiment that is not illustrated, cap 48 could be a plate that extends outwardly to and connects directly with annular channel 38. In any embodiment of the device, central base 44 and cap 48 are axially centered and vertically separated.

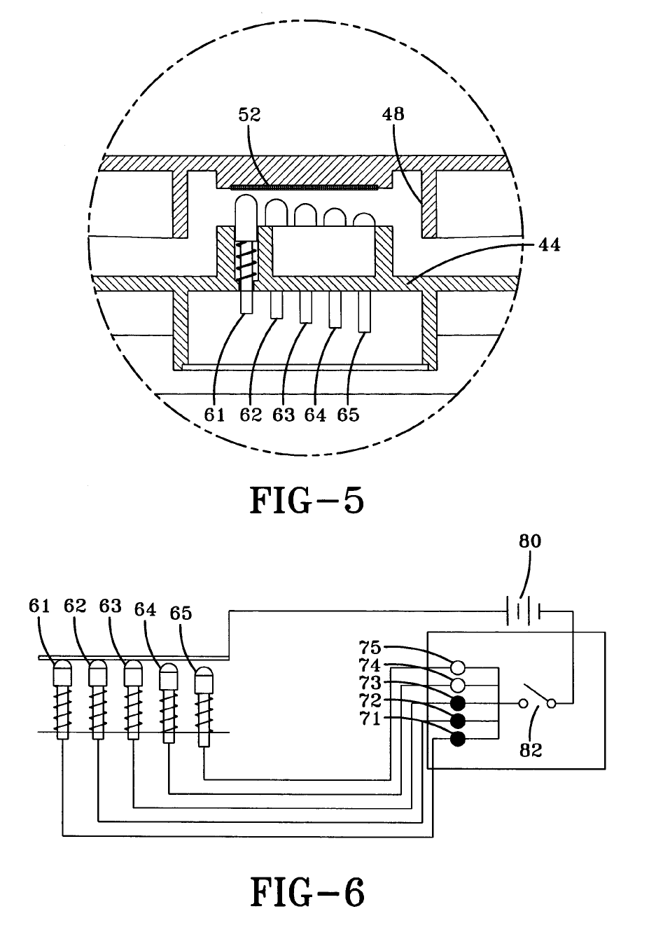

Other than the resilient suspension provided in the stanchions, cap 48 and central base 44 move independently of each other. In the embodiment shown in FIGS. 5 and 6, one example of a means for distinguishing between at least two fill levels of the vessel is shown. In the example, this means comprises a conductive contact plate 52 on a lower surface of the cap 48 and a plurality of electrical contact elements that are resiliently seated in the central base 44.

The operation of the electrical contact elements is illustrated schematically in FIGS. 5 and 6. In the depicted embodiment, five contact elements 61, 62, 63, 64 and 65, are shown. In a very minimal configuration, a single contact element 61 would be used, but such a configuration would only indicate whether the vessel needs to be refilled, and there would be no assurance to a user that the device is operating. Therefore, even in this minimal configuration, it would be expected that two indicators would be used, a first indicator 71 associated with the first contact element 61 and a “test” indicator that would light upon making a circuit. In the illustrated embodiment, there are five indicator means 71, 72, 73, 74 and 75, with each contact element connected as a part of an electrical circuit with a corresponding contact element. A typical indicator means would be a light-emitting diode (“LED”), due to its size, low power requirement and other consideration. Also a part of each circuit is a power means 80, shown in FIG. 6 as a three volt battery and the contact plate 52, which is positioned above each electrical contact element 61, 62, 63, 64 and 65. A switch 82 may be used to extend the life of the power means 80 when the system is not in use. Contact plate 52 is associated with the vessel-supporting member 20 by way of cap 48 and moves upwardly or downwardly as the weight on the vessel-supporting member changes. The contact elements 61-65 are arranged at different heights below the contact plate 52, so as more weight bears upon the contact plate, it makes electrical contact sequentially with contact elements 61, 62, 63, 64 and 65, which are mounted as a part of the base plate at increasing distances below the contact plate. In FIG. 6, contact elements 61 through 63 are in contact with contact plate 52, so indicator means 71 through 73 are illuminated.

In a typical embodiment, the only elements of the circuit contained in the cap 48 and the central base 44 will be the contact plate 52 and the electrical contacts 61-65. Conventional wiring, preferably in one of the radial arms, will communicate these elements with the power source and switch, which are preferably positioned in the base 20.

In a nominal 20 pound propane tank, the vessel itself weighs about 8.2 kg, and it can hold approximately 9.6 kg of propane when “full.” In a display panel associated with the five indicator means, the lighting of indicator 71 would indicate that there is less than about 1.2 kg of propane in the tank, that is, the tank weighs less than about 9.4 kg. When the propane weight is between 1.21 and 3.6 kg, indicator lights 71 and 72 should be illuminated, indicating “¼ full”. When the propane weight is between 3.61 and 6.0 kg, indicator lights 71-73 should be illuminated, indicating “½ full”. When the propane weight is between 6.01 and 8.4 kg, indicator lights 71-74 should be illuminated, indicating “¾ full”. Above 8.41 kg of propane in tank, all five indicators should be illuminated, indicating the tank to be “full.” Obviously, the tank scale can be calibrated so that different numbers of indicators may be used, or a larger or smaller capacity tank is accommodated.

Although the accuracy of the device 10 depends upon a proper selection of the spring 23 that urges the vessel-supporting member 30 upwardly from base 20 and the proper placement of electrical contact members 61, 62, 63, 64 and 65 relative to contact plate 52, this is a relatively simple adjustment that can be made by one of skill in this art.

It will also be understood that while the exemplary embodiment disclosed uses a plurality of electrical contact elements 61-65 to cause selective illumination of a plurality of illumination means 71-75, there are other means known to one of ordinary skill in this art to provide a similar signal as to the fill level of the tank. Just to present one example, a resistive or inductive strip could be used to provide a signal from which a micro-processor would send a signal to an appropriate number of illumination means.

As indicated above, a panel for display of the illumination means 71, 72, 73, 74 and 75 can be arranged on the base member 20, as on stanchion 22′, or it can be arranged remotely from the base member, such as on a control panel of the grill or other device with which the device is used. In particular, if the device 10 is used in association with a propane tank for a recreational vehicle, it would be common to provide a display of the tank fill level inside the vehicle, so an occupant would not have to go outside the vehicle to check the fill level.

It will be readily apparent that the device 10 may be manufactured from a polymeric material, especially an injection-moldable material such as a high-impact polystyrene.

While the embodiment shown and described herein is directed at use with a propane tank, such as that useful with a barbecue grill, the device can be used with a variety of products where the fill level of the vessel is not immediately obvious. These products would include pressurized gas cylinders, especially oxygen tanks used by medical patients or gas cylinders used in chemical laboratories. The device is also useful with beer kegs or with tanks of carbonated beverage.

While not shown in FIG. 1, some embodiments of the device 10 will be adapted, particularly in the base member 20, to be securely mounted horizontally onto a barbecue grill, a recreational vehicle, or other piece of equipment where the product in the tank will be used.

What is claimed is:

1. A device for measuring and displaying a relative fill level of a vessel, comprising:
a base, comprising an annular body with at least three stanchions, symmetrically spaced around a circumference of the annular body and a plurality of radially-inwardly-extending arms, each stanchion having a vertical slot and each of the radially inwardly extending arms meeting at an axial base member;
a vessel-supporting member having a plurality of radially-outwardly extending arms and a means for supporting a vessel atop the vessel-supporting member, each arm adapted at a distal end thereof to be received and retained in one of the vertical slots;
a plurality of means for resiliently supporting the vessel-supporting means radially inwardly from and above the base, the number of resilient support means corresponding to the number of vertical slots;
a means for distinguishing at least two levels of vertical separation between the base and the vessel-supporting member; and
a means for displaying at least two fill levels of the vessel, based upon the vertical separation distinguished.

2. The device of claim 1, wherein:
the displaying means comprises a switch to selectively make and break an electrical circuit for displaying the fill level measurement.

3. The device of claim 1, wherein:
each resiliently supporting means comprises a spring, the ends of which connect an interior of one of the stanchions to the distal end of one of the radially-outwardly extending arms.

4. The device of claim 1, wherein:
one of the stanchions is provided with a surface for at least one of: displaying the fill level of the vessel or connecting a remote fill level display device to the fill level distinguishing means.

5. The device of claim 1, wherein:
the vessel-supporting means comprises an annular concave surface for receiving a convex surface of the vessel.

6. The device of claim 1, wherein:
the vessel-supporting means comprises a channel for receiving a cylindrical base of the vessel.

7. The device of claim 2, wherein:
each resiliently supporting means comprises a spring, the ends of which connect an interior of one of the stanchions to the distal end of one of the radially-outwardly extending arms.

8. The device of claim 1, wherein:
the vertical separation distinguishing means comprises a plurality of electrical contacts resiliently positioned at different distances from a contact plate.

9. The device of claim 8, wherein:
the displaying means comprises an electrical circuit that is made by each of the each of the electrical contacts and the contact plate through contact, the circuit resulting in illumination of an indicator corresponding to the electrical contact.

10. The device of claim 9, wherein:
the indicators are light-emitting diodes.

11. The device of claim 9, wherein:
the circuit further comprises a touch switch that is normally open and closes to make the circuit only when activated by a user.
